(19) United States
(12) Patent Application Publication
(10) Pub. No.: US 2003/0105395 A1
(43) Pub. Date: Jun. 5, 2003

(54) Vessel-feeding pulmonary nodule candidate generation

(76) Inventors: Li Fan, Plainsboro, NJ (US); Jianzhong Qian, Princeton Junction, NJ (US); Guo-Qing Wei, Plainsboro, NJ (US)

Correspondence Address: Siemens Corporation, Intellectual Property Department, 186 Wood Avenue South, Iselin, NJ, 08830, US

(21) Appl. No.: 10/008,133

(22) Filed: Dec. 5, 2001

(51) Int. Cl. A61B 5/05

(52) U.S. Cl. 600/425

(57) ABSTRACT

A system and method for automatically generating potentially vessel-feeding pulmonary nodule candidates from multi-detector, thin-slice high resolution computed tomography images include a volume examination unit for providing a plurality of images defining a lung volume and examining the lung volume to generate a list of seed objects; a volume of interest generator for selecting a seed from the list and defining a volume of interest comprising the seed within the lung volume; a seed examination unit for extracting a structure of interest comprising the seed from the volume of interest, analyzing the structure of interest by automatically quantifying features therein, and updating the list of seed objects to exclude all unexamined seed objects contained in the current structure of interest under examination; and a candidate generator for generating a candidate from the structure of interest if its features meet preset criteria and providing geometric characteristics of the candidate to other algorithms for detecting pulmonary nodules.

### CROSS-REFERENCE TO RELATED APPLICATION

This application is related to the disclosure of co-pending Attorney Docket No. 8706-542 (2001 E15413US) entitled “Vessel-Feeding Pulmonary Nodule Detection By Volume Projection Analysis”, commonly assigned and concurrently filed herewith, the disclosure of which is incorporated by reference herein in its entirety.

### BACKGROUND

Pulmonary or lung cancer is currently a leading cause of cancer death. Early recognition of cancer-related pulmonary nodules may provide the greatest chance to prevent deaths due to lung cancer. Non-invasive, high-resolution, thin-slice, multi-slice or multi-detector computed tomography (“CT”) scanners are capable of providing detailed imaging data on anatomical structures. Therefore, non-invasive early recognition of pulmonary nodules from CT images holds great promise. Unfortunately, although vessel-feeding pulmonary nodules are more likely to be malignant compared with solitary ones, and of important clinical value, their accurate recognition from CT images is highly labor-intensive, technically challenging, and requires the careful attention of trained specialists.

### SUMMARY

These and other drawbacks and disadvantages of the prior art are addressed by a system and method for automatically generating potentially vessel-feeding pulmonary nodule candidates from multi-detector, thin-slice, high resolution computed tomography images.

The system and method include a volume examination unit for providing a plurality of images defining a lung volume and examining the lung volume to generate a list of seed objects; a volume of interest generator for selecting a seed object from the list and defining a volume of interest comprising the seed object within the lung volume; a seed examination unit for extracting a structure of interest comprising the seed object from the volume of interest, analyzing the structure of interest by automatically quantifying features therein, and updating the list of seed objects to exclude all unexamined seed objects contained in the current structure of interest under examination; and a candidate generator for generating a candidate from the structure of interest if its features meet preset criteria and providing geometric characteristics of the candidate to other algorithms for detecting pulmonary nodules.

These and other aspects, features and advantages of the present disclosure will become apparent from the following description of exemplary embodiments, which is to be read in connection with the accompanying drawings.

### BRIEF DESCRIPTION OF THE DRAWINGS

The present disclosure teaches an approach to finding suspicious structures of interest, or nodule candidates, that could potentially be pulmonary nodules, including vessel-feeding pulmonary nodules and solitary nodules.

FIG. 1 shows a block diagram of a system for automatically generating pulmonary nodule candidates from CT images according to an illustrative embodiment of the present disclosure;

FIG. 2 shows a flow diagram illustrating a method for automatically generating nodule candidates from CT images according to an illustrative embodiment of the present disclosure;

FIGS. 3, 4 and 5 show diagrams illustrating examples of seed object generation according to an illustrative embodiment of the present disclosure; and

FIGS. 6, 7 and 8 show diagrams illustrating examples of generated nodule candidates according to an illustrative embodiment of the present disclosure.

### DETAILED DESCRIPTION OF PREFERRED EMBODIMENTS

The present disclosure teaches a system and method for automatically generating vessel-feeding pulmonary nodule candidates from non-invasive, high-resolution, thin or multi-slice computed tomography (“CT”) images. The generated candidates are preferably subsequently supplied as input to a refined nodule detection procedure or nodule verification system, such as, for example, the system and method described in the above-referenced co-pending application entitled “Vessel-Feeding Pulmonary Nodule Detection By Volume Projection Analysis”.

Lung nodules can be classified into three major sub-categories including solitary nodules, nodules attached to chest walls, and nodules attached to vessels or vessel-feeding nodules. Among these, nodules attached to chest walls are eye-catching and relatively easy to recognize for radiologists. Anatomical structures of solitary nodules are relatively simple and usually sphere-like. Nodules attached to vessels, however, are much more difficult to recognize. This is because there is no obvious structural information that can easily catch radiologists' attention for further examination. Meanwhile, nodules with vessel-feeding morphology normally are more likely to be malignant compared with solitary ones, and of important clinical value.

A few approaches can be used to determine whether a suspicious structure may be a vessel-feeding nodule. Such approaches include template matching and three-dimensional rendering. However, without the initial findings of such suspicious structures of interest, all the approaches mentioned above can be very time-consuming, inefficient and result in an undesirably high rate of false positive diagnoses.

The present disclosure describes a novel approach for finding suspicious structures of interest, or nodule candidates, which may be used as the inputs for further processing so that other nodule detection schemes, such as, for example, one or more of the template matching and three-dimensional rendering methods, can be used to detect the nodules more accurately, efficiently, and precisely. The nodule candidate generation method described herein can be easily integrated as a screening function with other nodule recognition schemes in order to yield improved throughput and accuracy.

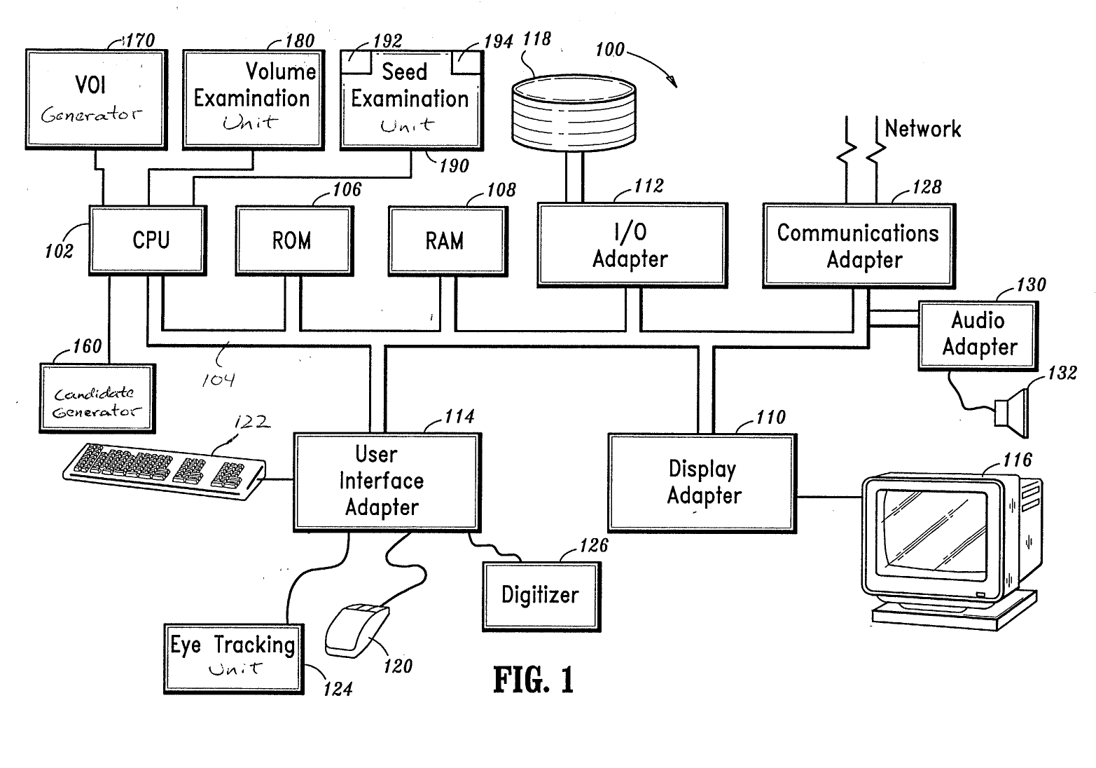

FIG. 1 shows a block diagram of a system 100 for automatically generating pulmonary nodule candidates from CT images, according to an illustrative embodiment of the present disclosure. The system 100 includes at least one processor or central processing unit (“CPU”) 102 in signal communication with a system bus 104. A read only memory (“ROM”) 106, a random access memory (“RAM”) 108, a display adapter 110, an I/O adapter 112, and a user interface adapter 114 are also in signal communication with the system bus 104.

A display unit 116 is in signal communication with the system bus 104 via the display adapter 110. A disk storage unit 118, such as, for example, a magnetic or optical disk storage unit, is in signal communication with the system bus 104 via the I/O adapter 112. A mouse 120, a keyboard 122, and an eye tracking unit 124 are also in signal communication with the system bus 104 via the user interface adapter 114. The mouse 120, keyboard 122, and eye-tracking unit 124 are used to aid in the generation of suspicious regions in a digital medical image.

A volume of interest (“VOI”) generator 170, a volume examination unit 180, a candidate generator 160, and a seed examination unit 190 that includes a segmentation unit 192 and a classifier 194 are also included in the system 100 and in signal communication with the CPU 102 and the system bus 104. While the VOI generator 170, the volume examination unit 180, the candidate generator 160, and the seed examination unit 190 that includes the segmentation unit 192 and the classifier 194 are illustrated as coupled to the at least one processor or CPU 102, these components are preferably embodied in computer program code stored in at least one of the memories 106, 108 and 118, wherein the computer program code is executed by the CPU 102.

The system 100 may also include a digitizer 126 in signal communication with the system bus 104 via a user interface adapter 114 for digitizing a CT image of the lungs. Alternatively, the digitizer 126 may be omitted, in which case a digital CT image may be input to the system 100 from a network via a communications adapter 128 in signal communication with the system bus 104, or via other suitable means as understood by those skilled in the art.

As will be recognized by those of ordinary skill in the pertinent art based on the teachings herein, alternate embodiments are possible, such as, for example, embodying some or all of the computer program code in registers located on the processor chip 102. Given the teachings of the disclosure provided herein, those of ordinary skill in the pertinent art will contemplate various alternate configurations and implementations of the volume examination unit 180, the VOI generator 170, the seed examination unit 190, the segmentation unit 192, the classifier 194, the candidate generator 160, as well as the other elements of the system 100, while practicing within the scope and spirit of the present disclosure.

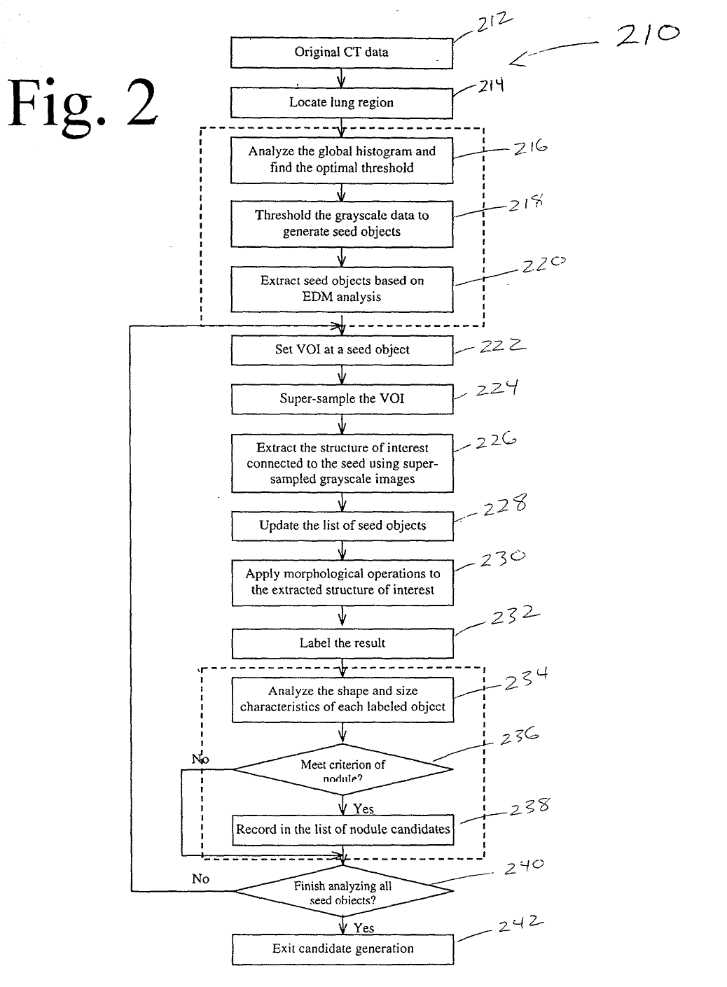

As shown in FIG. 2, a flow diagram illustrates a method 210 for automatically generating pulmonary nodule candidates from CT images according to an illustrative embodiment of the present disclosure. A general description of the present disclosure with respect to FIG. 2 will introduce the basic concepts, while subsequent description will detail various aspects of the disclosure with reference to the function blocks of FIG. 2.

The CT image data is loaded in function block 212, and preprocessing is carried out to locate the pulmonary volume in function block 214 by the lung segmentation unit 180 of FIG. 1, excluding the volume beyond the chest wall. A global histogram is analyzed in function block 216 in order to find an optimal threshold, the threshold is applied to grayscale data in function block 218 to include all the significant anatomical structures, and Euclidean Distance Map (“EDM”) techniques are applied in function block 220 in a way that can quickly exclude non-nodule structures from further relatively slow examinations and extract the seed objects, which are included in a list of seed objects. These seed objects represent the significant anatomical structures, including pulmonary nodule candidates, big vessels, and other tissues. Anatomical structures that may be considered as seed objects are preferably pre-specified.

The boundaries of a volume of interest (“VOI”) are defined according to the CT data by the VOI generator 170 of FIG. 1 at function block 222. In particular, the VOI is set up to traverse through the lung volume during nodule candidate generation. For each move of the VOI, the system preferably determines a local histogram of intensity within the VOI and from the histogram computes the adaptive threshold values for segmenting the VOI to obtain seed objects. If necessary, the VOI is super-sampled at function block 224 in order to achieve equivalent resolutions in all three dimensions.

In function block 226, a structure of interest comprising the seed object is extracted in correspondence with the super-sampled grayscale images, and the list of seed objects is updated in function block 228 to exclude all unexamined seed objects included in the current extracted structure of interest. In function block 230, morphological operations are applied to the structure of interest in order to classify the structure of interest, and the result is labeled in function block 232.

The seed examination unit 190 of FIG. 1 analyzes the shape and size characteristics of each labeled object at function block 234, and decision block 236 determines whether each labeled object meets the criteria for a nodule candidate. If a given object meets the criteria for a nodule candidate, it is recorded in the list of nodule candidates at function block 238 by the candidate generator 160 of FIG. 1. Decision block 240 determines whether all seed objects have been analyzed. If all seed objects have been analyzed, the candidate generation process is exited at function block 242; if not, control is passed back to function block 222 for processing of the next seed object.

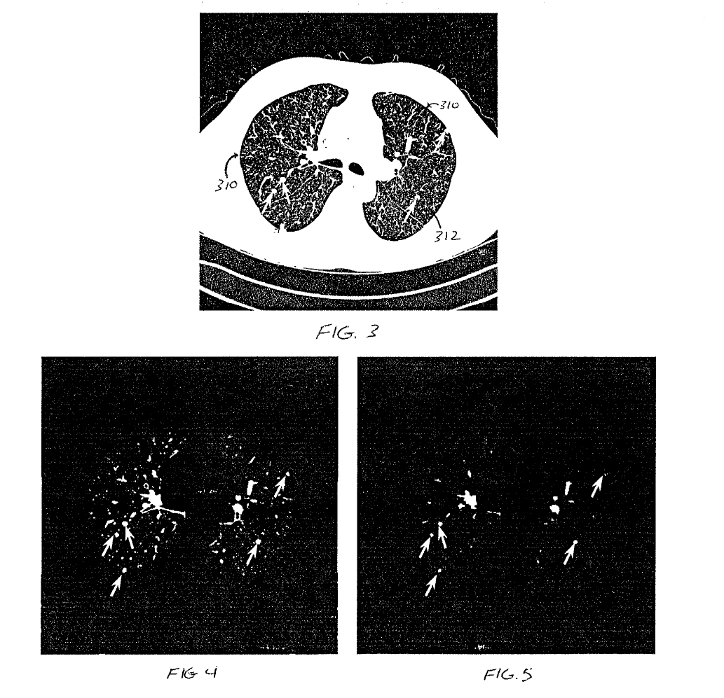

FIGS. 3, 4 and 5 show an exemplary sequence of the generation of seed objects and nodule candidates according to an illustrative embodiment of the method of FIG. 2. In particular, FIG. 3 illustrates an original CT image of a lung 310. FIG. 4 shows seed objects 314 generated by function block 218 of FIG. 2. FIG. 5 shows seed objects 316 generated by function block 220 of FIG. 1. In these FIGs., seed objects 312, 314 and 316 are represented by the bright opacities, and confirmed nodules are indicated for reference by bright arrows. The number of objects 316 of FIG. 5 is dramatically reduced by applying Euclidean Distance Map (“EDM”) analysis on FIG. 4.

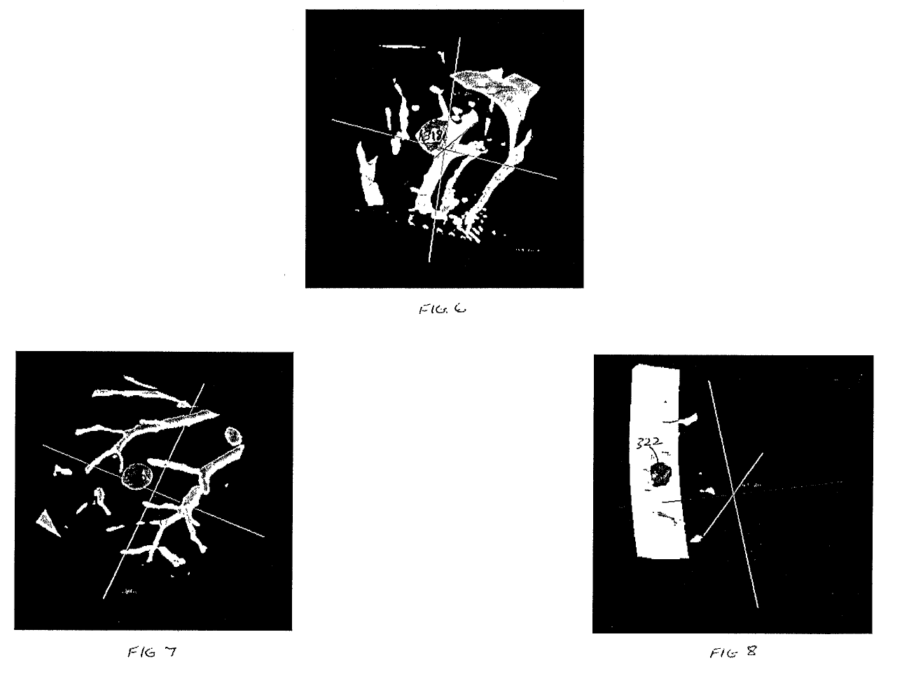

FIGS. 6, 7 and 8 show some examples of nodule candidates generated by the method of FIG. 2. FIG. 6 shows a candidate 318 of the vessel-feeding nodule type. FIG. 7 shows a candidate 320 of the solitary nodule type. FIG. 8 shows a candidate 322 of the chest wall-attaching nodule type.

Returning now to FIG. 2, the seed objects are examined to generate pulmonary nodule candidates by the seed examination unit 190 of FIG. 1 at function block 234. In particular, for each seed's corresponding structure extracted at function block 226, function blocks 230, 234 and 236 are performed to examine and classify the structure.

At function block 226, a segmentation method based on an analysis of the local histogram is applied to the seed objects by the segmentation unit 192 of FIG. 1 in order to extract the structure in accordance with three-dimensional connectivity properties. The intensity and geometric features of the extracted structure are computed in function block 230, where the structure is described by intensity and geometric parameters such as, for example, position, diameter, volume, circularity, sphericity, mean and standard deviation of intensity.

The extracted structure is classified as a nodule candidate or non-nodule by the classifier 194 of FIG. 1 at decision block 236, based on multiple criteria and/or a priori knowledge about pulmonary nodule candidates and other proximate structures. The criteria may include, for example, properties such as intensity, volume and shape, as determined in function blocks 230 and/or 234. If the extracted structure is categorized as a nodule candidate at decision block 236, then the nodule candidate is automatically recorded at function block 238.

The pulmonary nodule candidates may be visualized on the display 116 of FIG. 1, such as shown in FIGS. 3, 4, 5, 6, 7 and 8. For each candidate, further processing may be directed to visualization and heuristic candidate verification. The visualization is particularly desirable when nodule candidates are attached to pulmonary vessels.

The pulmonary nodule surface is rendered, such as is shown in FIGS. 6, 7 and 8. A three-dimensional free rotation is provided to facilitate the study of the structure of interest and its relationship to the connected vessels and the surrounding structures. The nodule candidates are analyzed to render a classification decision for output at function block 238, such as, for example, to a user, storage medium, and/or the like.

In operation, the function blocks 214 and 228 facilitate efficient processing by reducing extraneous analysis. First, the pulmonary volume is located in function block 214 so that the search volume for suspicious structures is narrowed. Then, in function block 228, all the seed objects that are included in the current structure of interest are excluded from the seed object list. In this way, non-nodule structures, such as vessels and the airway tree, are examined once and then excluded from further study.

In an illustrative approach for making the recognition decision in combination with the teachings of the present disclosure, the pulmonary nodule features such as, for example, the shape, are automatically quantified and the recognition decision is made by a candidate verification system, such as, for example, that disclosed in the above-referenced co-pending application entitled “Vessel-Feeding Pulmonary Nodule Detection By Volume Projection Analysis”, which has been incorporated by reference herein in its entirety.

The nodule candidates are documented and recorded at function block 238. In particular, the analysis results such as, for example, measurements and analysis results of function blocks 218, 220, 226, 230 and 234, are automatically saved for future use. This is very useful for follow-up examination and treatment monitoring, as well as candidate verification.

Computational efficiency is an important factor for evaluating a pulmonary nodule candidate generation method. When CT scans are performed and hundreds of slice images need to be examined, this issue becomes very significant. The computational complexity is reduced, as discussed above, for example, by extracting the pulmonary area from the original images in function block 214 so that the lung volume to be examined is narrowed. On two-dimensional (“2-D”) axial image slices, for example, pulmonary volumes are usually dark areas with some bright structures inside, while surrounding tissues, such as the chest wall and heart, appear to be much brighter regions that are interconnected. Clear boundaries between the pulmonary area and non-pulmonary area can be defined as follows. A global threshold is set by automatically analyzing the histogram of the entire volumetric data at function block 216 to optimally distinguish pulmonary tissues that contain air content from other solid tissues that have higher mass density, such as muscle, bone, and vessels. Thresholding is then applied at function block 218 to each two-dimensional image slice. The chest wall interconnection with the heart is usually the largest structure labeled, and can be easily identified. The pulmonary volume is then obtained by excluding beyond the chest wall, without excluding any seed objects embedded therein.

In function blocks 216, 218 and 220, computation time is further saved. On 2-D axial slices, the global optimal threshold is applied so that only important anatomical structures, such as nodules, vessels, are kept. However, there will be a large number of objects for which the intensity is above the threshold, and most of these are normal anatomy. To reduce the number of objects that need detailed examination, Euclidean Distance Map (“EDM”) is applied. Only structures that have EDM values above a preset threshold are kept for further analysis. In this way, most of the linear shaped structures such as vessels are excluded and the number of seed objects is reduced dramatically. An example can be seen by comparing the number of bright opacities in FIG. 4 and FIG. 5, respectively.

In function block 224, a VOI is generated after applying the global threshold and extracting the pulmonary area on every two-dimensional slice. With respect to function block 220, binary pixel regions that are high-valued or “on” in this data represent significant anatomical structures, including nodule candidates, blood vessels, bronchial walls, and other tissues, and serve as initial seed objects to examine the structures of interest. Note that since the threshold is set to achieve global optimization, anatomical structures may be broken into pieces after segmentation. Multiple seed objects contained in the binary image data may therefore represent the same anatomical structure. Accordingly, function block 228 updates the list of seed objects by removing all references to seed objects already considered, so that duplicative processing is avoided.

With respect to function block 226, segmentation of target structures plays an important role in the whole scheme. Quantitative measurements and further classification are based on the segmentation results for the structures of interest.

An intensity threshold for segmentation is dynamically chosen based on the curvature extrema analysis on the local histogram in the VOI. The shape and size of the VOI are defined according to the CT data characteristics. Once the local threshold has been determined, a three-dimensional region growing method is applied to segment the target structure in function block 226. It begins with the seed under consideration; all the points that have intensity values higher than the threshold and that are connected to the known part will be added into the segmentation result.

However, multiple seed objects determined in correspondence with the global threshold may belong to the same anatomical structure at the local level. Computation would be inefficient if every seed were examined and the same structure had to be repeatedly segmented. Therefore, the binary volumetric data that contains all the seed objects is updated after segmentation at function block 228 in order to reduce computational redundancy. Seed objects will be turned off if they are determined to be connected to the current seed under examination. In this way, non-nodule structures, such as, for example, vessels and the airway tree, are examined once and then excluded from future study. This has been shown to dramatically reduce the number of seed objects on each slice and to save computational time.

With respect to function block 236, the structure is classified as a nodule candidate or a non-nodule structure by measuring and analyzing geometric properties in function block 234 that characterizes the structure, after it has been extracted. While illustrative properties are described herein, including diameter, volume, sphericity, mean intensity value and standard deviation of intensity, other properties may be employed in place of, or in addition to the exemplary illustrative properties as will be recognized by those of ordinary skill in the pertinent art.

Although the parameters of diameter and volume are not independent of each other and contain some redundant information, both are still measured in illustrative examples because it is still common for radiologists to use diameter to express the size of a pulmonary nodule during initial screenings but meanwhile use volume to determine the growth rate.

Sphericity is the three-dimensional counterpart of circularity that is used to measure compactness, and is defined as the fraction of a structure's volume to the volume of a sphere that encompasses it. This parameter characterizes the three-dimensional shape of a structure of interest. Although nodule candidates and blood vessels may both have circular shapes on two-dimensional slices, their three-dimensional shapes are quite different. Pulmonary nodule candidates are sphere-like with high compactness, while blood vessels are tube-like, with very low compactness. It has been found that circularity and sphericity are very useful in separating pulmonary nodule candidates from vessels. Cutoff values of circularity and sphericity are empirically set. Structures that are larger than a given diameter, such as, for example, 2 mm, and have sphericity measurements higher than the chosen cutoff values, will be considered as pulmonary nodule candidates, and their positions recorded.

In operation, the present disclosure teaches automatically generating pulmonary nodule candidates from CT images for subsequent input to other refined detection methods so that radiologists and physicians can be freed from the heavy burden of reading through multitudes of image slices. An advantage of the present disclosure is the provided sensitivity to pulmonary nodule candidates while maintaining low false-positive rates. Usually, pulmonary nodule candidates appear in slice images as nearly circular-shaped opacities, which are similar to cross-sections of vessels. Accordingly, most existing recognition methods using only 2D information have a high false-positive rate. The present disclosure solves this problem by incorporating a priori anatomical knowledge of pulmonary structures and making full use of the three-dimensional image information. Multiple criteria, including geometric and intensity criteria, are set up for categorizing the suspicious volumes of interest (“VOI”) as containing pulmonary nodule candidates or non-nodule structure. Furthermore, the segmentation and extraction methods of the present disclosure adjust the segmentation threshold based on local histogram analysis, which has particular advantages over prior approaches using only a global histogram analysis when used in the presence of the high noise typical in low-dose screening images.

The present disclosure is computationally efficient, and provides for a timely method of automatic candidate generation for use as input to other nodule recognition methods, so that an examining physician may review the results in a timely manner. The present disclosure teaches functions associated with a pulmonary nodule candidate generation method that are compatible with nodule recognition and/or verification methods to facilitate the examination of patient data by physicians. Such functions include surface rendering of structures of interest, parameter measurement, documentation of suggested nodule candidates, and so forth. These and other features and advantages of the present disclosure may be readily ascertained by one of ordinary skill in the pertinent art based on the teachings herein.

It is to be understood that the teachings of the present disclosure may be implemented in various forms of hardware, software, firmware, special purpose processors, or combinations thereof. Most preferably, the teachings of the present disclosure are implemented as a combination of hardware and software. Moreover, the software is preferably implemented as an application program tangibly embodied on a program storage unit. The application program may be uploaded to, and executed by, a machine comprising any suitable architecture. Preferably, the machine is implemented on a computer platform having hardware such as one or more central processing units (“CPU”), a random access memory (“RAM”), and input/output (“I/O”) interfaces. The computer platform may also include an operating system and microinstruction code. The various processes and functions described herein may be either part of the microinstruction code or part of the application program, or any combination thereof, which is executed via the operating system. In addition, various other peripheral units may be connected to the computer platform such as an additional data storage unit and a printing unit.

It is to be further understood that, because some of the constituent system components and method function blocks depicted in the accompanying drawings are preferably implemented in software, the actual connections between the system components or the process function blocks may differ depending upon the manner in which the present disclosure is programmed. Given the teachings herein, one of ordinary skill in the pertinent art will be able to contemplate these and similar implementations or configurations of the present disclosure.

An embodiment of the above-described approach automatically generates pulmonary nodule candidates, especially vessel-feeding nodule candidates, from multi-detector, thin-slice, high-resolution computed tomography images. The embodiment generates nodule candidates in a two-stage fashion. First, the method of the embodiment quickly and roughly examines the lung volume and excludes most of the non-nodule structures from further detailed examinations. To do so, Euclidean Distance Map (“EDM”) techniques are applied in a way that can quickly exclude small to middle-sized vessels and generate a list of seed objects. Second, more detailed examination is applied to further exclude the non-nodule structures and generate a list of nodule candidates. This is done by applying morphological operations to analyze the geometric characteristics of the above-obtained seed objects. Only those objects meeting certain preset criteria are considered as pulmonary nodule candidates and output.

Thus, the method of the instant embodiment includes examining the lung volume to generate a list of seed objects by applying EDM techniques in a specific way; defining a volume of interest comprising the seed within the lung volume; analyzing the structure of interest by automatically quantifying features therein; and generating candidate from the structure of interest if the features meet some preset criteria.

There are several particularly innovative features present in the instant embodiment. First, it is very computationally efficient because of the two-stage analysis fashion. A large number of non-nodule structures are excluded by the fast EDM analysis and only a limited number of structures need detailed or relatively slow morphological analysis. Second, the method can provide important geometric information about the nodule candidates for other nodule detection algorithms, such as, for example, an optional final detection scheme based on template matching and/or three-dimensional rendering techniques. The reduction in the number of nodule candidates, along with the provision of important geometric characteristics, helps to substantially reduce the risk of producing false positives in the optional final detection schemes and therefore improves the overall detection accuracy.

Although the illustrative embodiments have been described herein with reference to the accompanying drawings, it is to be understood that the present disclosure is not limited to those precise embodiments, and that various changes and modifications may be affected therein by one of ordinary skill in the pertinent art without departing from the scope or spirit of the present disclosure. All such changes and modifications are intended to be included within the scope of the present disclosure as set forth in the appended claims.

What is claimed is:

1. A method for automatically generating pulmonary nodule candidates from images, the method comprising:
providing a plurality of images defining a lung volume;
examining the lung volume to generate a list of seed objects;
selecting a seed from the list;
defining a volume of interest comprising the seed;
extracting a current structure of interest comprising the seed object from the volume of interest;
analyzing the current structure of interest by automatically quantifying features therein;
updating the list of seed objects to exclude unexamined seed objects contained in the current structure of interest from further examination;
recording the current structure of interest as a candidate in the candidate list if the automatically quantified features of the current structure of interest meet preset criteria; and
providing at least one of the automatically quantified features of the candidate to a nodule verification system.

2. A method as defined in claim 1 wherein said examining comprises:
excluding small to middle-sized non-nodule structures from further detailed examination; and
generating the list of seed objects.

3. A method as defined in claim 1 wherein said provided at least one of the automatically quantified features comprises geometric characteristics.

4. A method as defined in claim 1 wherein said images comprise at least one of high-resolution, thin-slice and multi-slice computed tomography images.

5. A method as defined in claim 1 wherein said candidate comprises a pulmonary nodule candidate.

6. A method as defined in claim 5 wherein said pulmonary nodule candidate comprises a vessel-feeding pulmonary nodule.

7. A method as defined in claim 1, further comprising:
examining said candidate to recognize a pulmonary nodule therefrom.

8. A method as defined in claim 1, further comprising:
displaying said candidate; and
analyzing said candidate by recalling the quantified features of the corresponding structure to provide an automatic recognition decision for said candidate.

9. A method as defined in claim 1, further comprising:
super-sampling the volume of interest to obtain equivalent resolutions in three dimensions;

10. A method as defined in claim 4 wherein examining the lung volume to obtain seed objects comprises:
determining a global histogram of intensity inside the lung volume;
thresholding image slices to keep only important anatomical structures; and applying Euclidean Distance Mapping to exclude linear-shaped structure such as vessels and reduce the number of seed objects.

11. A method as defined in claim 1 wherein extracting a structure of interest comprises:
adaptively adjusting a local threshold value based on a local histogram analysis of the volume of interest; and
defining anatomical structures based on three-dimensional connectivity and intensity information corresponding to the local threshold.

12. A method as defined in claim 1 wherein said defining a volume of interest comprises:
defining a shape and a size of the volume of interest.

13. A method as defined in claim 1 wherein said examining the lung volume to obtain seed objects comprises:
determining an adaptive segmentation threshold value based upon an analysis of the global histogram.

14. A method as defined in claim 1 wherein said analyzing the structure of interest comprises:
computing intensity and geometric features of the segmented, anatomical structures.

15. A method as defined in claim 14 wherein said intensity and geometric features comprise position, volume, circularity, sphericity, mean intensity and standard deviation of intensity.

16. A method as defined in claim 1, wherein said generating candidate comprises:
recording a segmented, anatomical structure for further evaluation.

17. A method as defined in claim 1 wherein at least one of said analyzing the structure of interest and said generating candidate comprises:
excluding non-nodule structures from further evaluation.

18. A method as defined in claim 8 wherein said displaying said candidate comprises:
rendering surfaces of said candidate to provide three-dimensional visualization with the freedom of 3D rotation.

19. A method as defined in claim 8, further comprising:
storing the automatic recognition decision.

20. A method as defined in claim 7 wherein said examining said candidate comprises:
receiving an external recognition decision for said candidate from a user.

21. A method as defined in claim 19, further comprising:
storing the external recognition decision.

22. A system (100) for automatically generating candidates from images, the system comprising:
a volume examination unit (180) for at least one of providing a plurality of images defining a lung volume, examining the lung volume to generate a list of seed objects from the lung volume;
a volume of interest generator (170) in signal communication with the volume examination unit (180) for at least one of selecting a seed from the list, defining a volume of interest comprising the seed within the lung volume, and super-sampling the volume of interest to obtain comparable resolutions in three dimensions;
a seed examination unit (190) in signal communication with the volume of interest generator (170) for at least one of extracting a structure of interest comprising the seed from the volume of interest, analyzing the structure of interest by automatically quantifying features therein, and updating the list of seed objects to exclude all seed objects contained in the current structure of interest; and
a candidate generator (160) in signal communication with the seed examination unit (190) for generating candidate from the structure of interest if the features meet some preset criteria.

23. A system (100) as defined in claim 22 wherein said images comprise high-resolution, thin-slice, multi-slice, computed tomography images.

24. A system (100) as defined in claim 22 wherein said candidate comprises a pulmonary nodule candidate.

25. A system (100) as defined in claim 24 wherein said pulmonary nodule candidate comprises a vessel-feeding pulmonary nodule, or a pulmonary nodule of the other two types (solitary or attached to chest wall).

26. A system (100) as defined in claim 22, further comprising:
a CPU (102) in signal communication with said candidate generator (160) for examining said candidate.

27. A system (100) as defined in claim 26, further comprising:
a display adapter (110) in signal communication with the CPU (102) for displaying said candidate; and
an I/O adapter (112) in signal communication with the CPU (102) for recalling the quantified features of the corresponding structure of said at least one candidate to provide an automatic recognition decision for said candidate.

28. A system (100) as defined in claim 26, further comprising:
a user interface adapter (114) in signal communication with the CPU (102) for at least receiving an external recognition decision for said candidate from a user.

29. A system for automatically generating candidates from images, the system comprising:
means for providing a plurality of images defining a lung volume;
means for examining the lung volume to generate a list of seed objects;
means for selecting a seed from the list;
means for defining a volume of interest comprising the seed within the lung volume;
means for extracting a structure of interest comprising the seed from the volume of interest;
means for analyzing the structure of interest by automatically quantifying features therein;
means for updating the list of seed objects to exclude all seed objects contained in the structure of interest; and
means for generating candidate from the structure of interest if the features meet some preset criteria.

30. A program storage device readable by machine, tangibly embodying a program of instructions executable by the machine to perform method steps for automatically generating candidates from images, the method steps comprising:
providing a plurality of images defining a lung volume;
examining the lung volume to generate a list of seed objects;
selecting a seed from the list;
defining a volume of interest comprising the seed;
extracting a current structure of interest comprising the seed object from the volume of interest;
analyzing the current structure of interest by automatically quantifying features therein;
updating the list of seed objects to exclude unexamined seed objects contained in the current structure of interest from further examination;
recording the current structure of interest as a candidate in the candidate list if the automatically quantified features of the current structure of interest meet preset criteria; and
providing at least one of the automatically quantified features of the candidate to a nodule verification system.
